(19) United States
(12) Patent Application Publication
(10) Pub. No.: US 2002/0099099 A1
(43) Pub. Date: Jul. 25, 2002

(54) High molecular weight primary aliphatic alcohols obtained from natural products and uses thereof

(76) Inventors: William R. Gamble, Longmont, CO (US); Zhengjie Liu, Superior, CO (US); David T. Bailey, Boulder, CO (US); Pedro P. Perez, Miami, FL (US); Dean P. Stull, Longmont, CO (US); Steven L. Richheimer, Westminster, CO (US); Rebecca L. Nichols, Broomfield, CO (US); Rod Lenoble, Westminster, CO (US)

Correspondence Address: Steven C. Petersen, Hogan & Hartson, LLP, Suite 1500, 1200 17th Street, Denver, CO, 80202, US

(21) Appl. No.: 09/949,285

(22) Filed: Sep. 7, 2001

Related U.S. Application Data
(63) Continuation-in-part of application No. 09/845,043, filed on Apr. 27, 2001.
Application No. 09/845,043 is a continuation-in-part of application No. 09/337,339, filed on Jun. 21, 1999.
(60) Provisional application No. 60/236,515, filed on Sep. 29, 2000.

(51) Int. Cl. A61K 31/045; C07C 31/00

(52) U.S. Cl. 514/724; 568/840

(57) ABSTRACT

The present invention relates to a naturally obtained mixture of higher molecular weight primary aliphatic alcohols which contain 20 to 34 carbon atoms. This invention also relates to the process for obtaining the alcohol mixture by extraction and purification with organic solvents from a natural product, such as beeswax with and without saponification of the natural product. The alcohol mixture obtained from beeswax has enhanced purity and contains a mixture of alcohols having 20, 22, 24, 26, 27, 28, 30, 32 and 34 carbon atoms. The alcohol mixture is useful in pharmaceutical compositions, foodstuffs and dietary supplements and is effective for lowering cholesterol levels so that it is effective in treating hypercholesterolemia.

### CROSS-REFERENCE TO OTHER APPLICATIONS

This patent application is a continuation-in-part application of U.S. patent application No. 09/845,043, filed Apr. 27, 2001, which is a continuation-in-part of U.S. Pat. No. 6,225,354, filed Jun. 21, 1999, and entitled “High Molecular Weight Primary Aliphatic Alcohols Obtained From Beeswax and Pharmaceutical use Thereof” and this patent application claims priority from Provisional Application No. 60/236,515 filed on Sep. 29, 2000, and entitled “High Molecular Weight Primary Aliphatic Alcohols Obtained From Beeswax and Uses Thereof.”

### BACKGROUND OF THE INVENTION

1. Field of the Invention

The present invention pertains to a biologically active mixture of high purity, high molecular weight primary straight chained aliphatic alcohols from a naturally occurring source such as beeswax. More particularly the invention pertains to a mixture of high purity, high molecular weight primary straight chained aliphatic alcohols that is obtained from saponified and/or unsaponified beeswax by liquid extraction wherein the resulting alcohols in the mixture contain 20 to 34 carbon atoms.

2. Description of the State of Art

All kinds of waxes, and more especially beeswax, have always been a matter of interest. This has been the case not only because of their industrial applications but also because of their chemical composition. The amount of beeswax in honey ranges between 0.9% to 1.13%, depending on the methods used to separate the wax from the honey. This wax contains esters, hydrocarbons, free fatty acids, free alcohols and a long list of minor compounds.

The natural mixture of primary straight chained aliphatic alcohols obtained from beeswax has been studied by several authors to learn about its composition and main features. The obtaining of different mixtures of alcohols from various waxes has been reported. (J. A. Lamberton, et al., Australian Journal of Chemistry, 13:261-268 (1959); A. Horn and J. S. Martic, Journal of Science Food and Agriculture, 10:571 (1957); Kreger, (1948); Wimbero, (1904); and Mitsui and Col, (1942)). These studies suggest a method for obtaining fatty alcohols based on the homogeneous saponification with alcoholic potassium hydroxide, followed by the reesterification of the saponified material, and separation of the alcohols.

Another method reports an extraction of the natural alcohol mixture through a high efficiency vacuum. The high vacuum wax distillation for the chemical isolation of mixed derivatives and the extraction of the remaining wax is done using petroleum ether. The solvent evaporates and the remaining solids are acetylated for further purification through alumina chromatography. Finally, through alkaline hydrolysis, alcohols are obtained and then recrystallized in ethanol, showing a fusion point ranging from 62 to 82° C.

Blood-lipid lowering effects of a natural mixture of straight chain aliphatic alcohols have been demonstrated by several authors: (F. Liu, et al., “Active Constituents Lowering Blood-Lipid in Beeswax”, Zhongguo Zhong Yao Za Zhi, 21(9):553-4, 576 (1996)); (H. Sho, et al., “Effects of Okinawa Sugar Cane Wax and Fatty Alcohols on Serum and Liver Lipids in the Rats”, J. Nutri Vitaminol, 30(6):553-559 (1984)); (S. Kato, K. Hamatani, et al., “Octacosanol Effects Lipid Metabolism in Rat Fed on a High Fat Diet”, Br J Nutr, 73(3):433-441 (1995)); (Kabiry et al., “Tissue Distribution of Octacosanol in Liver and Muscle of Rats After Serial Administration”, Ann Nutr Metab, 39(5):279-284 (1995)). Many investigational studies based on clinical studies using the natural mixtures of straight chain aliphatic alcohols have been published.

A procedure for obtaining a natural mixture of straight chain higher aliphatic primary alcohols from animal and vegetable waxes (a natural source wax) is also known in the prior art. This prior art procedure is based on the extraction of alcohol mixtures with fluid extractant in the sub and supercritical states between 20 and 100° C. Selective extraction can be carried out with this procedure but when this is applied to beeswax it is only possible to obtain between 10% to 15% of a C-20 to C-34 alcohol mixture.

Another project (S. Inaa, K. Furukama, T. Masui, K. Honda, J. Ogasawara, and G. Tsubikamoto, “Process for Recovering Primary Normal Aliphatic Higher Alcohols”, JP 60-119514 (1996)) proposed a very similar extraction method applied to waxes that is based on fluids (CO₂ with ethylene) in sub and supercritical states.

There are different commercial dietary supplements, foods and drugs to aid in the lowering of total blood cholesterol (lowering lipid, LDL, and cholesterol levels) which are considered as effective, safe and well tolerated but most produce different adverse side effects. Since lipid-lowering therapy must be chronically administered, safety and tolerableness are very important for their definitive acceptance. Although many products from different sources exist in the market such as β-sitosterol, garlic, bile acid binders, fibric acid derivatives, HMG-Co A reductase inhibitors and nicotinic acid, etc., the methods of use and the dosage recommendations for these products are not effective for the reduction of cholesterol to the desired levels. In addition, the drugs that are used for the lowering of cholesterol have various adverse side effects.

### SUMMARY OF THE INVENTION

The method and resulting composition of the present invention begins by initially subjecting beeswax to a homogenous phase saponification step after which the saponified beeswax is recovered, dried, and ground to a particle mesh size of 100-500 microns. Alternatively, the saponification step may be skipped entirely and the unsaponified beeswax may be dried and ground to a particle mesh size of 100-200 microns.

Next, the particles of beeswax (saponified or unsaponified) are placed into a conventional solid-liquid extractor and a hot organic solvent is introduced and contacted with the beeswax particles. The resulting solution is hot filtered to remove the solids. The extract is then maintained within the temperature range of 2° C.-10° C. causing the alcohols to solidify and form a suspension. The suspension is filtered and the solids are recovered and air dried. The solid obtained from the drying step is sent to a purifier where it is contacted with and dissolved in another hot organic solvent. This solution is then hot filtered and cooled. The solids are again collected and dried by vacuum. The dried solids obtained from the purification step are contacted with another hot organic solvent which dissolves the solids. This solution is hot filtered and chilled to resolidify the solids. The solids are collected, dried and powdered.

The final product contains a mixture of higher primary aliphatic alcohols from 20 to 34 carbon atoms comprising 1-eicosanol, 1-docosanol, 1-tetracosanol, 1-hexacosanol, 1-heptacosanol, 1-octacosanol, 1-triacontanol, 1 -dotriacontanol and 1-tetratriacontanol having the following quantitative composition:

| | | |
|---|---|---|
| 1-eicosanol | C-20 | 0-5% |
| 1-docosanol | C-22 | 0-5% |
| 1-tetracosanol | C-24 | 12-27% |
| 1-hexacosanol | C-26 | 13-28% |
| 1-heptacosanol | C-27 | 0-5% |
| 1-octacosanol | C-28 | 15-25% |
| 1-triacontanol | C-30 | 25-40% |
| 1-dotriacontanol | C-32 | 5-15% |
| 1-tetratriacontanol | C-34 | 0-5% |

and may be used as is or reformulated for administration to humans and animals to reduce and prevent hyper-cholesterolemic diseases, cholesterol, coronary heart disease (heart attacks and strokes), inflammation or immunoregulatory diseases, cardiovascular diseases, and/or neurodegenerative disorders. The daily dosage is established between 1 to 100 mg per day (preferably 3 to 20 mg) and is intended for ingestion in any type or form of foodstuff, capsule, tablet or liquid form.

### BRIEF DESCRIPTION OF THE DRAWINGS

The accompanying drawings, which are incorporated in and form a part of the specification, illustrate the preferred embodiment of the present invention, and together with the description serve to explain the principles of the invention.

In the Drawings

FIG. 1 is a flow diagram depicting the saponification step of the process according to the present invention.

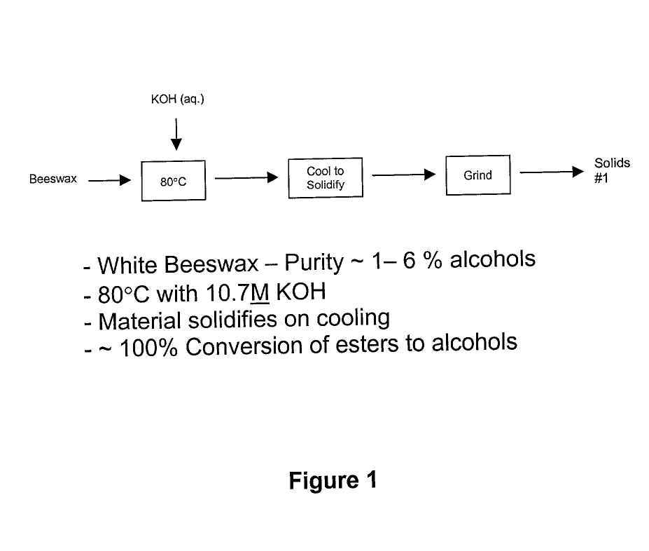

FIG. 2 is a reaction diagram depicting the theoretical yields as a result of the saponification step of the process according to the present invention.

FIG. 3 is a flow diagram depicting the extraction step of the process according to the present invention.

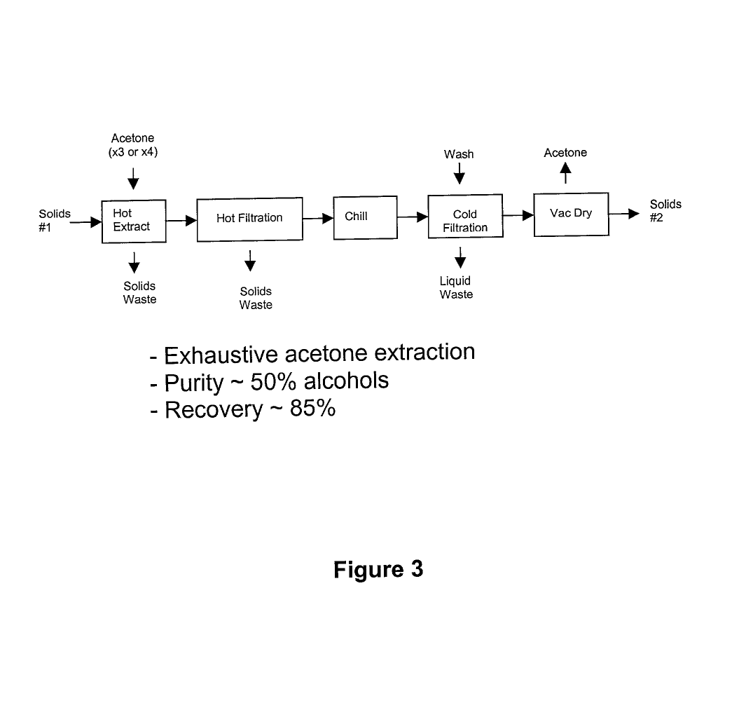

FIG. 4 is a flow diagram depicting the first purification step of the process according to the present invention.

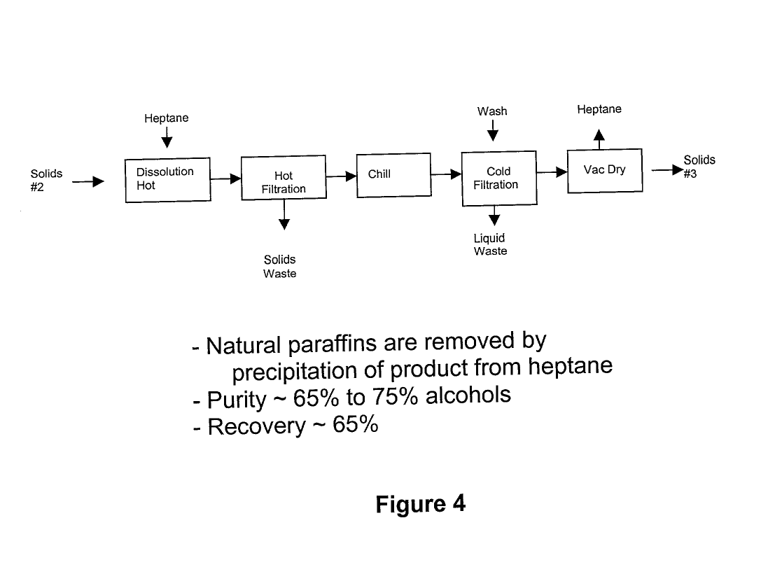

FIG. 5 is a flow diagram depicting the second purification step of the process according to the present invention.

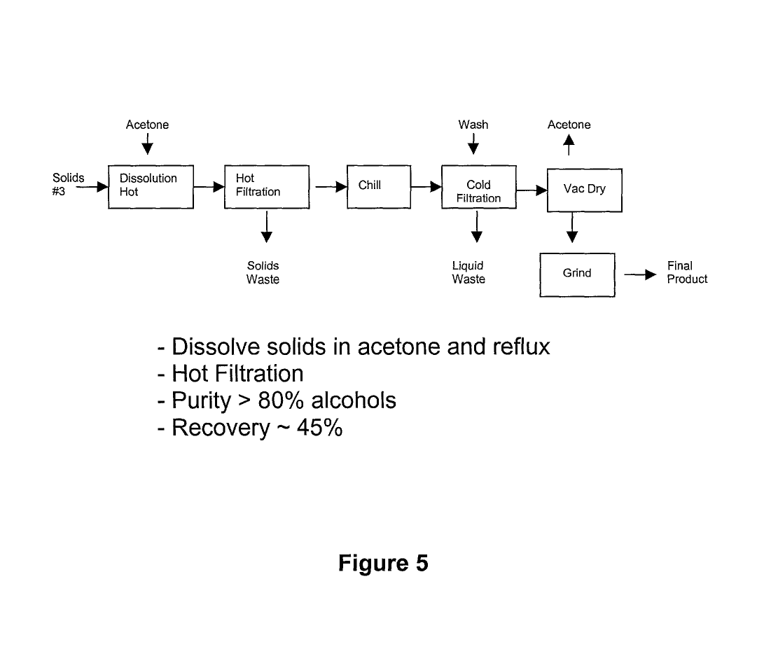

### DETAILED DESCRIPTION OF INVENTION AND PREFERRED EMBODIMENTS

The composition of the present invention is a mixture of primary high molecular weight straight chain aliphatic alcohols (hereinafter referred to as higher aliphatic primary alcohols) obtained by saponifying, extracting and purifying from beeswax of varying purity, the natural mixture of straight chain primary aliphatic alcohols (higher aliphatic primary alcohols). The beeswax is initially subjected to a homogenous phase saponification step after which the saponified beeswax is dried and ground to a particle mesh size of 100-500 microns. Alternatively, unsaponified beeswax, of varying purity, may be used as the starting material and is initially dried and ground to a particle mesh size of 100-200 microns. Next, the particles of saponified or unsaponified beeswax are placed into a conventional solid-liquid extractor and a hot organic solvent is introduced and contacted with the beeswax particles. The suspension is then hot filtered to remove any waste solids.

The resulting extract is maintained within the temperature range of 2° C.-10° C. causing the alcohols to solidify and form a suspension. The suspension is filtered and the solids are recovered and air dried. The dried solid obtained from the drying step is then sent to a purifier where it is contacted with and dissolved in another hot solvent and hot filtered. This solution is then cooled and the solids collected and dried by vacuum. The dried solids obtained from the purification step are then contacted with another hot organic solvent, which dissolves the solids. This solution is then hot filtered and chilled and the solids collected, dried, and powdered.

The final product, whether derived from saponified or unsaponified beeswax, contains a mixture of higher primary straight chained aliphatic alcohols from 20 to 34 carbon atoms comprising 1-eicosanol, 1-docosanol, 1-tetracosanol, 1-hexacosanol, 1-heptacosanol, 1-octacosanol, 1-triacontanol, 1-dotriacontanol, and 1-tetratriacontanol having the following quantitative composition:

| | | |
|---|---|---|
| 1-eicosanol | C-20 | 0-5% |
| 1-docosanol | C-22 | 0-5% |
| 1-tetracosanol | C-24 | 12-27% |
| 1-hexacosanol | C-26 | 13-28% |
| 1-heptacosanol | C-27 | 0-5% |
| 1-octacosanol | C-28 | 15-25% |
| 1-triacontanol | C-30 | 25-40% |
| 1-dotriacontanol | C-32 | 5-15% |
| 1-tetratriacontanol | C-34 | 0-5% |

This product has been developed for use in lowering LDL and total cholesterol and improving LDL/HDL ratios.

It has been discovered that the use of beeswax as the source of the alcohol mixture offers numerous benefits which cannot be obtained when sugar cane wax is used as the source. For example, many factors are associated with purity levels of sugar cane wax which are difficult to control and thus it is difficult to obtain a reliable source of sugar cane wax which meets the requirements for consistently obtaining a uniform pure product.

In particular, the purity level of sugar cane wax is influenced by the sugar cane variety, age of the plant, soil and climate conditions where the sugar cane is grown and the level and type of fertilizer used to grow the sugar cane. In addition, the type of operational procedure used to extract the wax from the husk can influence the level of purity. None of these factors are significant when beeswax is used as the source for the alcohol mixture. Furthermore, minor variations in beeswax characteristics can be corrected by blending selected waxes which meet certain criteria with respect to parameters as further described herein.

Furthermore, preferred levels of certain operational parameters in the saponification (optional), extraction and purification process have been discovered which lead to further enhancement of the purity level of the isolated alcohols and enhanced percent recovery of the alcohols from the beeswax. These operational parameters include the following: particle size of the solid (i.e., the particle size of the beeswax or saponified beeswax), alkali concentration, relationship between solid and liquid (i.e., solid:liquid ratio), temperature range, fluid regimen, crystallization regimen, hot filtration regimen, centrifuge regimen and contact time. The preferred levels of these parameters were established under experimental design in the laboratory as well as at the pilot plant and industrial levels.

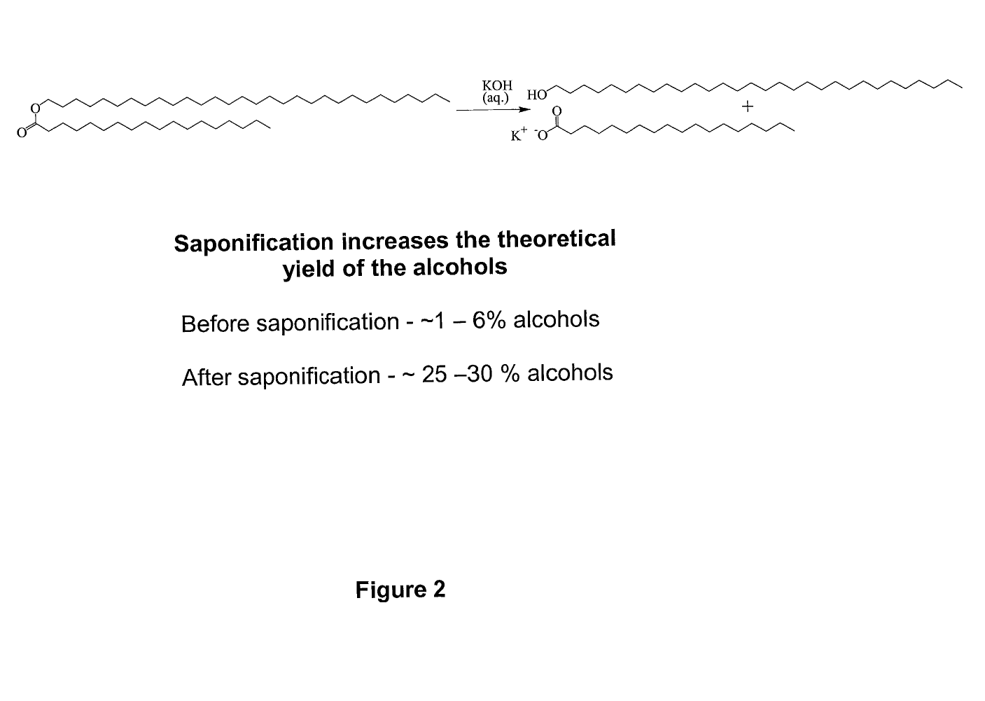

The procedure for a homogeneous phase saponification process of beeswax in the present invention consists of melting the beeswax at a temperature of 80° C.-100° C., to which an aqueous solution of potassium hydroxide (10.7 M) is added with continuous stirring at 40-100 rpm. The saponification process is continued for 3 hours with continuous stirring at 80 rpm. It has been determined that each pound of wax requires between 38 grams and 47 grams of potassium hydroxide for complete saponification. The homogeneous phase saponification process of beeswax in the present invention increases the theoretical yield of the alcohols as shown in FIG. 2. Yield is the only parameter effected when the beeswax is saponified or left unsaponified.

In a preferred embodiment the beeswax or saponified beeswax is cooled and ground to achieve a particle size of 100-500 microns in diameter, preferably 250 microns in diameter. The particles of ground wax are placed into a conventional solid-liquid extractor. A hot organic solvent extractant is also introduced into the extractor for contact with the particles of beeswax contained therein. Preferably acetone is used as the extractant. However, other examples of suitable solvents, such as but not limited to pentanone, toluene, benzene, ethanol, heptane, propanol, isopropanol, ethyl acetate, methanol, hexane, n-butanol, trichloroethane, methyl ethyl ketone, 1,2-dichloroethane, dichloromethane, chloroform and mixtures thereof may also be used. The ratio of beeswax particles to liquid extractant is from 1:4 to 1:8, preferably 1:6. The extraction is conducted for 3 to 7 hours, preferably 4 hours, within a temperature range of 50° C. -60° C., preferably 56° C. The beeswax particles are preferably agitated during the extraction procedure, for example by use of a rotating agitator to agitate the particles while in contact with the solvent. Advantageously the agitator is rotated at 40-100 rpm, preferably 80 rpm. During the extraction procedure the alcohols become solubilized in the extractant thus leaving a waxy residue. This extraction is repeated two times. Upon completion of the extraction, the extractant containing the alcohols dissolved therein is removed from the waxy residue.

Next the extract is introduced into a chamber for solidifying the alcohols. The alcohols are advantageously solidified by reducing the temperature of the extract in the chiller to form solids in the extractant (e.g., an alcohol in organic solvent solution). Preferably the temperature in the chiller should be uniform. An agitator may be provided within the crystallizer to assure a uniform temperature therein. The agitator is rotated at 40-80 rpm, preferably 60 rpm. The temperature during solidification is maintained within the range of 2° C.-10° C., preferably 6° C. for about 18 hours.

The suspension or mixture obtained from the chiller is vacuum filtered or centrifuged to recover the solids. The solids are routinely washed with chilled solvent. Centrifugation takes place by centrifuging the mixture or suspension at 1200-1400 rpm for about 2 hours. During centrifugation the particles may be washed with a spray of clean extraction solvent to remove contaminating material which may be contained in the mother liquor.

The clean solid mixture obtained from the centrifugation step is recovered and dried. Vacuum drying may be used. A pressure of 400 millibars at a temperature up to 50° C. may be used during the vacuum drying step.

The dried solid obtained from the vacuum drying step is sent to a purifier where it is contacted with another hot organic solvent, which is preferably heptane. Other hot organic solvents such as, but not limited to pentanone, toluene, benzene, ethanol, heptane, propanol, isopropanol, ethyl acetate, methanol, hexane, n-butanol, trichloroethane, methyl ethyl ketone, 1,2-dichloroethane, dichloromethane, chloroform and mixtures thereof may also be used. The alcohol solids are dissolved in the hot heptane to form a solution. The solution is hot filtered before being introduced into a chiller for solidification. The same type of chiller may be used in the solidification step as was used in the initial solidification from the acetone. The temperature of the heptane solution is kept uniform by agitating with an agitator at 40-80 rpm, preferably 60 rpm. The temperature during resolidification is maintained at 15° C.-25° C., preferably 20° C. The resolidification step forms a mixture or suspension of solids in the heptane solvent.

The suspension or mixture of solids in the heptane is introduced into a filtration or centrifugation device where the solids are separated in the same manner as the solids were recovered from the acetone solvent. During this second filtration step, the solids can be washed with clean solvent (heptane).

The particles obtained from the second filtration step are then recovered and dried. Vacuum drying may be used. A pressure of 400 millibars at a temperature up to 50° C. may be used during the vacuum drying step.

The dried solid obtained from the heptane purification step is sent to a purifier where it is contacted with another hot organic solvent which is preferably acetone at about 40 parts acetone to 1 part solid. Other hot organic solvents such as, but not limited to pentanone, toluene, benzene, ethanol, heptane, propanol, isopropanol, ethyl acetate, methanol, hexane, n-butanol, trichloroethane, methyl ethyl ketone, 1,2-dichloroethane, dichloromethane, chloroform and mixtures thereof may also be used. The solids are dissolved in the acetone. The hot acetone solution is passed through a hot filtration system. The filtered acetone solution is then introduced into a chiller for resolidification. The solidification is performed under similar conditions as the initial solidification of the alcohols from the acetone solution. Thus the same type of chiller may be used in the solidification step. The temperature of the hot acetone solution is kept uniform by agitating at 40-80 rpm, preferably 60 rpm. The temperature during resolidification is maintained at 2° C.-10° C., preferably 6° C. The resolidification step forms a mixture or suspension of solids in the acetone solvent.

The suspension or mixture of solids in the acetone is introduced into a filter, where it is filtered in the same manner that the solid suspension or mixture was filtered from the first acetone solvent. During this third filtration step, the crystals are washed with clean solvent (acetone). The washed particles obtained from the third filtration step are recovered and dried. Vacuum drying may be used. A pressure of 400 millibars at a temperature up to 35° C. may be used during the vacuum drying step.

After the particles are dried, they are then ready to be formulated into a conventional pharmaceutical formulation such as tablets, capsules, etc., for administration.

The yield (i.e., percent recovery by weight of alcohols with respect to the weight of alcohols in saponified beeswax) attained ranges about 40% with purity ranges from 80% to 99%. The natural mixture obtained contains alcohols ranging from 20 to 34 carbon atoms, with a melting point between 61°-65° C. The natural mixture of straight chain aliphatic alcohols obtained by this process may be analyzed through gas chromatography using a fused silica capillary column.

The procedure of this invention for obtaining the natural mixture of higher primary molecular weight aliphatic alcohols from beeswax has some advantages compared to other prior art procedures.

Advantages of this invention (when using saponified beeswax) are related to the practical yields (10%-15% by weight) compared with the previously reported results with yields lower than 5%. Another advantage of the procedure relates to the purity that can be obtained (80%-99%) which is significantly higher than the purity in the prior art methods. Thus, the method of the present invention is simple and appropriate for large scale production.

The pharmaceutical composition, foodstuffs, and dietary supplements formulated with the natural mixture of higher aliphatic primary alcohols of this invention may be administered to humans and animals. The daily dosage of this natural mixture obtained from beeswax to be used for the reduction and prevention of hyper-cholesterolemic diseases, cholesterol, coronary heart disease (heart attacks and stroke), inflammation or immunoregulatory diseases, cardiovascular disease and neurodegenerative disorders is established between 1 to 100 mg per day (preferably 3 to 20 mg) and is intended for ingestion in any type or form of foodstuff, capsule, tablet or liquid form.

The below-described examples further describe the invention and preferred embodiments thereof.

### EXAMPLES

Saponification:

Seven kg of wax were heated using a water bath at 80° C.-85° C. for 2.5 hours until the wax was completely melted. Once the wax was completely melted, 1.22 L of 10.7 M KOH in water were added dropwise over 30 minutes while continuing the stirring and heating. The mixture was held at temperature for 3 hours with stirring. After 3 hours the saponified wax was poured into trays and dried in a 60° C.-65° C. vacuum oven. The 7.54 kg of dried saponified wax were then ground using a Cuisinart blender.

Extraction:

The saponified solids were placed in a 72 L round bottom flask along with 45 L of acetone. The acetone was brought to reflux and held there for 3 hours. The heat was then removed and the solids allowed to settle. The extract was decanted through cheesecloth into 5-gallon buckets. An additional forty-five liters of fresh acetone were added to the solids in the round bottom flask and the extraction and decantation repeated to generate a total of 3 extracts. The extracts were chilled at 4° C.-8° C. for 40-45 hours, and the solids recovered by filtration using Buchner funnels and Whatman #4 filter paper. The 2.78 kg of collected solids were air dried for 48 hours.

Heptane Purification:

The acetone extract solids were dissolved in 60 L of heptane by holding the solution at 60° C.-70° C. for 2 hours with stirring. The hot solution was poured into five-gallon buckets which were sealed and cooled to 50° C. for 27 hours. The solutions were then filtered using Buchner funnels equipped with 50 μm porosity monofilament nylon cloth. The solids in the Buchner funnels were rinsed with an additional 1.5 L of fresh heptane and the solids dried in a vacuum oven at 45° C. and 15 inches Hg to provide 1.9 kg of heptane solids.

Acetone Purification:

The heptane solids were extracted with 36 L of acetone at reflux for 2 hours. This extract was hot filtered and put into sealed buckets. An equal volume of fresh acetone was added to the solids and the extraction repeated for an additional 2 hours. The extracts were chilled at approximately 5° C. for 42 hours and then the solid precipitate collected on Buchner funnels using Whatman #4 filter paper. The solids in the Buchner funnels were rinsed with an additional 1.5 L of fresh acetone and dried in a 35° C. vacuum oven for 17 hours to provide 0.942 kg of product.

The final product described above was used in an open label, single center study to evaluate the effectiveness and tolerability of higher aliphatic primary alcohols at lowering LDL, total cholesterol, and improving LDL/HDL ratio, the results of which are summarized below, involving 14 subjects who took 10 mgs of higher aliphatic primary alcohols per day for 6 weeks. There were no controls on the subjects diets. All data are reported in mg/dL.

TABLE 1
CHOLESTEROL

| SUBJECT | T1 | T2 | % Chg | T3 | % Chg |
|---|---|---|---|---|---|
| 1 | 218 | 243 | 11% | 229 | 5% |
| 2 | 200 | 216 | 8% | 212 | 6% |
| 3 | 170 | 150 | −12% | 157 | −8% |
| 4 | 207 | 155 | −25% | 171 | −17% |
| 5 | 256 | 217 | −15% | 221 | −14% |
| 6 | 206 | 196 | −5% | 170 | −17% |
| 7 | 314 | 253 | −19% | 268 | −15% |
| 8 | 176 | 167 | −5% | 171 | 3% |
| 9 | 161 | 164 | 2% | 159 | −1% |
| 10 | 156 | 167 | 7% | 173 | 11% |
| 11 | 240 | N.T. | N/A | 220 | −8% |
| 12 | 212 | 185 | −13% | 184 | −13% |
| 13 | 260 | 212 | −18% | 224 | −14% |
| 14 | 231 | 208 | −10% | 241 | 4% |
| Average | 215 | 195 | −9% | 200 | −7% |
| p* | | 0.06 | | 0.36 | |

T1 = Baseline (before taking higher aliphatic primary alcohols)
T2 = 3 weeks of 10 mgs higher aliphatic primary alcohols per day
T3 = 6 weeks of 10 mgs higher aliphatic primary alcohols per day
N.T. = Not tested, N/A = Not applicable
*Comparison with baseline by Wilcoxon statistical analysis

TABLE 2
TRIGLYCERIDES

| SUBJECT | T1 | T2 | % Chg | T3 | % Chg |
|---|---|---|---|---|---|
| 1 | 162 | 143 | −12% | 211 | 30% |
| 2 | 64 | 100 | 56% | 63 | −2% |
| 3 | 97 | 128 | 32% | 109 | 12% |
| 4 | 428 | 203 | −53% | 300 | −30% |
| 5 | 198 | 108 | −45% | 213 | 8% |
| 6 | 96 | 111 | 16% | 49 | −49% |
| 7 | 270 | 238 | −12% | 204 | −24% |
| 8 | 68 | 74 | 9% | 67 | −1% |
| 9 | 69 | 92 | 33% | 68 | −1% |
| 10 | 82 | 109 | 33% | 108 | 32% |
| 11 | 48 | N.T. | N/A | 99 | 106% |
| 12 | 213 | 220 | 3% | 196 | −8% |
| 13 | 125 | 186 | 49% | 185 | 48% |
| 14 | 155 | 119 | −23% | 118 | −24% |
| Average | 148 | 141 | −5% | 142 | −4% |
| p* | | 1.00 | | | 0.86 |

T1 = Baseline (before taking higher aliphatic primary alcohols)
T2 = 3 weeks of 10 mgs higher aliphatic primary alcohols per day
T3 = 6 weeks of 10 mgs higher aliphatic primary alcohols per day
N.T. = Not tested, N/A = Not applicable
*Comparison with baseline by Wilcoxon statistical analysis

TABLE 3
HDL

| SUBJECT | T1 | T2 | % Chg | T3 | % Chg |
|---|---|---|---|---|---|
| 1 | 41 | 42 | 2% | 40 | −2% |
| 2 | 48 | 45 | −6% | 60 | 25% |
| 3 | 32 | 31 | −3% | 33 | 3% |
| 4 | 38 | 41 | 8% | 49 | 29% |
| 5 | 59 | 69 | 17% | 63 | 7% |
| 6 | 50 | 51 | 2% | 58 | 16% |
| 7 | 61 | 54 | −11% | 66 | 8% |
| 8 | 49 | 45 | −8% | 54 | 10% |
| 9 | 54 | 49 | −9% | 48 | −11 |
| 10 | 60 | 56 | −7% | 71 | 18% |
| 11 | 63 | N.T. | N/A | 62 | −2% |
| 12 | 42 | 35 | −17% | 41 | −2% |
| 13 | 42 | 34 | −19% | 38 | −10% |
| 14 | 61 | 57 | −7% | 63 | 3% |
| Average | 50 | 47 | −6% | 53 | 7% |
| p* | | 0.11 | | 0.08 | |

T1 = Baseline (before taking higher aliphatic primary alcohols)
T2 = 3 weeks of 10 mgs higher aliphatic primary alcohols per day
T3 = 6 weeks of 10 mgs higher aliphatic primary alcohols per day
N.T. = Not tested, N/A = Not applicable
*Comparison with baseline by Wilcoxon statistical analysis

TABLE 4
LDL

| SUBJECT | T1 | T2 | % Chg | T3 | % Chg |
|---|---|---|---|---|---|
| 1 | 144.6 | 172.4 | 19% | 147.0 | 2% |
| 2 | 139.2 | 151.0 | 8% | 139.0 | 0% |
| 3 | 118.6 | 93.0 | −22% | 102.0 | −14% |
| 4 | N/A | 73.4 | N/A | 62.0 | N/A |
| 5 | 157.4 | 126.0 | −20% | 115.0 | −27% |
| 6 | 136.8 | 122.8 | −10% | 100.0 | −27% |
| 7 | 199.0 | 151.4 | −24% | 161.0 | 19% |
| 8 | 113.4 | 107.2 | −5% | 104.0 | −8% |
| 9 | 93.2 | 96.6 | 4% | 97.0 | 4% |
| 10 | 79.6 | 89.2 | 12% | 80.0 | 1% |
| 11 | 167.4 | N.T. | N/A | 138.0 | −18% |
| 12 | 127.4 | 106.0 | −17% | 104.0 | −18% |
| 13 | 183.0 | 140.8 | −23% | 149.0 | −19% |
| 14 | 139.0 | 127.2 | −8% | 154.0 | 11% |
| Average | 138.0 | 120.0 | −13% | 118.0 | −15% |
| p* | | 0.11 | | 0.03 | |

T1 = Baseline (before taking higher aliphatic primary alcohols)
T2 = 3 weeks of 10 mgs higher aliphatic primary alcohols per day
T3 = 6 weeks of 10 mgs higher aliphatic primary alcohols per day
N.T. = Not tested, N/A = Not applicable
*Comparison with baseline by Wilcoxon statistical analysis

TABLE 5
Cardiovascular Disease Risk (CDR)**

| | | | | | |
|---|---|---|---|---|---|
| SUBJECT | T1 | T2 | % Chg | T3 | % Chg |

| | | | | | |
|---|---|---|---|---|---|
| 1 | 5.32 | 5.79 | 9% | 5.73 | 8% |
| 2 | 4.17 | 4.80 | 15% | 3.53 | −15% |
| 3 | 5.31 | 4.84 | −9% | 4.76 | −10% |
| 4 | 5.45 | 3.78 | −31% | 3.49 | −36% |
| 5 | 4.34 | 3.14 | −28% | 3.51 | −19% |
| 6 | 4.12 | 3.84 | −7% | 2.93 | −29% |
| 7 | 5.15 | 4.69 | −9% | 4.06 | −21% |
| 8 | 3.59 | 3.71 | 3% | 3.17 | −12% |
| 9 | 2.98 | 3.35 | 12% | 3.31 | 11% |
| 10 | 2.60 | 2.98 | 15% | 2.44 | −6% |
| 11 | 3.81 | N.T. | N/A | 3.55 | −7% |
| 12 | 5.05 | 5.29 | 5% | 4.49 | −11% |
| 13 | 5.92 | 6.24 | 5% | 5.89 | −1% |
| 14 | 3.79 | 3.65 | −4% | 3.83 | 1% |
| Average | 4.40 | 4.32 | −2% | 3.91 | −11% |
| p* | | 0.26 | | 0.01 | |

T1 = Baseline (before taking higher aliphatic primary alcohols)
T2 = 3 weeks of 10 mgs higher aliphatic primary alcohols per day
T3 = 6 weeks of 10 mgs higher aliphatic primary alcohols per day
N.T. = Not tested, N/A = Not applicable
*Comparison with baseline by Wilcoxon statistical analysis

As demonstrated by the data in the above tables, there was a 15% reduction in LDL-C that is statistically significant at 96.7% confidence level.

The 7% reduction in total cholesterol is not statistically significant. While the 3 week data had shown a 9% reduction and was statically significant at 94% confidence level, the 6 week data does not support this trend. The most likely explanation is that of diet, which is not controlled in this study.

It is important to note that with an uncontrolled diet it is not uncommon to see an increase in cholesterol and LDL over the course of the study. This has been demonstrated in the placebo group in other higher aliphatic primary alcohols studies, see Canetti, M., et. al., “A two-year study on the efficacy and tolerability of policosanol in patients with type II hyperlipoproteinemia”, Int. J. Clin. Pharm. Res., 15(4):159-165 (1995).

A 7% increase in HDL levels, 92% confidence level, was observed and was not unexpected. Prior studies in humans have shown an increase in HDL levels.

The CDR was reduced by 11%, 99% confidence level. This is due to the decrease cholesterol and increased HDL levels and was expected.

There was no improvement in triglycerides, and this is not unexpected. No prior studies on higher aliphatic primary alcohols have indicated a reduction in triglycerides.

The data look promising. Previous studies of higher aliphatic primary alcohols, sugar cane material, by Pones P., et. al., “Effects of successive dose increases of policosanol on the lipid profile of patients with type II hypercholesterolaemia and tolerability to treatment”, Int. J. Clin. Pharm. Res., 14(1):27-33 (1994), demonstrated an 8% reduction in total cholesterol in 8 weeks with 5 mg/day and a 14% reduction in total cholesterol in 16 weeks with 5 mg/day for the first 8 weeks and then 10 mg/day for the remaining 8 weeks. They also reported a 11% and 22% reduction in LDL from the same study. These data are consistent with the results of the current study and one would expect to see further reduction in both LDL and total cholesterol if the study duration were increased.

### Higher Aliphatic Primary Alcohols Analysis

All final acetone extracts were separately evaporated to dryness without exceeding 35° C. The samples were analyzed for total higher aliphatic primary alcohols content by GC-FID.

### Results

The percentage of higher aliphatic primary alcohols in the starting material (either saponified or unsaponified beeswax) was calculated based on the total higher aliphatic primary alcohols content in each acetone extract. Gas chromatography analyses for individual higher aliphatic primary alcohols percentages are listed in Table 6 below. The higher aliphatic primary alcohols found in saponified beeswax were as high as 30%.

TABLE 6

| | Yield | | | | | | | | | |
|---|---|---|---|---|---|---|---|---|---|---|
| | Total % of | PERCENTAGE OF EACH ALCOHOL | | | | | | | | |
| SOURCE | Alcohols | C20 | C22 | C24 | C26 | C27 | C28 | C30 | C32 | C34 |
| Saponified | 25-30 | 0 | 0 | 18 | 17 | 0 | 19 | 31 | 15 | 0 |
| Beeswax | | | | | | | | | | |
| Unsaponified | 1-6 | 0 | 0 | 14 | 14 | 0 | 18 | 33 | 20 | 0 |
| Beeswax | | | | | | | | | | |

The foregoing description is considered as illustrative only of the principles of the invention. Further, since numerous modifications and changes will readily occur to those skilled in the art, it is not desired to limit the invention to the exact construction and process shown as described above. Accordingly, all suitable modifications and equivalents may be resorted to falling within the scope of the invention as defined by the claims that follow.

The words “comprise,” “comprising”, “include,” “including,” and “includes” when used in this specification and in the following claims are intended to specify the presence of stated features, integers, components, or steps, but they do not preclude the presence or addition of one or more other features, integers, components, steps, or groups thereof.

The embodiments of the invention in which an exclusive property or privilege is claimed are defined as follows:

1. A method for isolating a mixture of primary aliphatic alcohols from non-alcoholic compounds contained in natural wax wherein said method comprises subjecting saponified beeswax to liquid extraction with a liquid organic extractant in which said alcohols are soluble; recovering said alcohol mixture from said extractant whereby said alcohol mixture is isolated from said non-alcoholic compounds contained in said saponified beeswax and said isolated alcohol mixture includes:

| | | |
|---|---|---|
| 1-eicosanol | C-20 | 0-5% |
| 1-docosanol | C-22 | 0-5% |
| 1-tetracosanol | C-24 | 12-27% |
| 1-hexacosanol | C-26 | 13-28% |
| 1-heptacosanol | C-27 | 0-5% |
| 1-octacosanol | C-28 | 15-25% |
| 1-triacontanol | C-30 | 25-40% |
| 1-dotriacontanol | C-32 | 5-15% |
| 1-tetratriacontanol | C-34 | 0-5%. |

2. The method of claim 1 wherein said liquid organic extractant is selected from the group comprising: acetone, toluene, benzene, ethanol, heptane, hexane, pentanone, methanol, propanol, isopropanol, ethyl acetate, ether, trichloroethane, methyl ethyl ketone, n-butanol, 1, 2-dichloroethane, dichloromethane, chloroform and mixtures thereof; and said isolated alcohol mixture has a purity level of 80-99% with respect to said non-alcoholic compounds contained in said beeswax.

3. A mixture of primary aliphatic alcohols isolated from beeswax; said mixture containing:

| | | |
|---|---|---|
| 1-eicosanol | C-20 | 0-5% |
| 1-docosanol | C-22 | 0-5% |
| 1-tetracosanol | C-24 | 12-27% |
| 1-hexacosanol | C-26 | 13-28% |
| 1-heptacosanol | C-27 | 0-5% |
| 1 -octacosanol | C-28 | 15-25% |
| 1-triacontanol | C-30 | 25-40% |
| 1-dotriacontanol | C-32 | 5-15% |
| 1-tetratriacontanol | C-34 | 0-5%. |

4. A pharmaceutical composition which comprises the mixture of claim 3 in combination with a pharmaceutically acceptable carrier, excipient or dilutant.

5. The composition of claim 4 in the form of a capsule, tablet, liquid or powder.

6. A method for treating or preventing hypercholesterolemia related diseases which comprises administering a pharmaceutically effective amount of the mixture of claim 4 to a human or mammal.

7. A method for reducing total cholesterol and LDL levels which comprises administering a pharmaceutically effective amount of the mixture according to claim 4 to a human or mammal.

8. A method for lowering LDL, total cholesterol, and improving LDL/HDL ratio which comprises administering the mixture of claim 4 in a pharmaceutically acceptable amount to an individual in need thereof.

9. A method for isolating a mixture of primary aliphatic alcohols from non-alcoholic compounds contained in natural wax wherein said method comprises subjecting unsaponified beeswax to liquid extraction with a liquid organic extractant in which said alcohols are soluble; recovering said alcohol mixture from said extractant whereby said alcohol mixture is isolated from said non-alcoholic compounds contained in said unsaponified beeswax and said isolated alcohol mixture includes:

| | | |
|---|---|---|
| 1-eicosanol | C-20 | 0-5% |
| 1-docosanol | C-22 | 0-5% |
| 1-tetracosanol | C-24 | 12-27% |
| 1-hexacosanol | C-26 | 13-28% |
| 1-heptacosanol | C-27 | 0-5% |
| 1-octacosanol | C-28 | 15-25% |
| 1-triacontanol | C-30 | 25-40% |
| 1-dotriacontanol | C-32 | 5-15% |
| 1-tetratriacontanol | C-34 | 0-5%. |

10. The method of claim 9, wherein said liquid organic extractant is selected from the group comprising: acetone, toluene, benzene, ethanol, heptane, hexane, pentanone, methanol, propanol, isopropanol, ethyl acetate, ether, trichloroethane, methyl ethyl ketone, butanol, 1,2-dichloroethane, dichloromethane, chloroform and mixtures thereof; and said isolated alcohol mixture has a purity level of 80-99% with respect to said non-alcoholic compounds contained in said unsaponified beeswax.

11. A mixture of primary aliphatic alcohols isolated from unsaponified beeswax; said mixture containing:

| | | |
|---|---|---|
| 1-eicosanol | C-20 | 0-5% |
| 1-docosanol | C-22 | 0-5% |
| 1-tetracosanol | C-24 | 12-27% |
| 1-hexacosanol | C-26 | 13-28% |
| 1-heptacosanol | C-27 | 0-5% |
| 1-octacosanol | C-28 | 15-25% |
| 1-triacontanol | C-30 | 25-40% |
| 1-dotriacontanol | C-32 | 5-15% |
| 1-tetratriacontanol | C-34 | 0-5%. |

12. A pharmaceutical composition which comprises the mixture of claim 11 in combination with a pharmaceutically acceptable carrier, excipient or dilutant.

13. The composition of claim 12 in the form of a capsule, tablet, liquid or powder.

14. A method for treating or preventing hypercholesterolemia related diseases which comprises administering a pharmaceutically effective amount of the mixture of claim 12 to a human or mammal.

15. A method for reducing total cholesterol and LDL levels which comprises administering a pharmaceutically effective amount of the mixture according to claim 12 to a human or mammal.

16. A method for lowering LDL, total cholesterol, and improving LDL/HDL ratio which comprises administering the mixture of claim 12 in a pharmaceutically acceptable amount to an individual in need thereof.
